(19) United States
(12) Patent Application Publication
(10) Pub. No.: US 2023/0285002 A1
(43) Pub. Date: Sep. 14, 2023

(54) SYSTEMS AND METHODS FOR ASSESSING INTERNAL LUMEN SHAPE CHANGES TO SCREEN PATIENTS FOR A MEDICAL DISORDER

(71) Applicant: Timothy Shine, Jacksonville, FL (US)

(72) Inventor: Timothy Shine, Jacksonville, FL (US)

(21) Appl. No.: 18/019,002

(22) PCT Filed: Jul. 30, 2021
(86) PCT No.: PCT/US2021/043966

§ 371 Date: Jan. 31, 2023

Related U.S. Application Data
(60) Provisional application No. 63/059,242, filed on Jul. 31, 2020.

(51) Int. Cl. A61B 8/08; A61B 8/06

(52) U.S. Cl. CPC A61B 8/5223; A61B 8/06; A61B 8/0891; A61B 8/488

(57) ABSTRACT

Systems and methods for assessing internal lumen shape changes to screen patients for a medical disorder or condition are described herein. Infectious diseases have stages from mild infection to severe infection. Each particular infectious organism and infectious state is expected to produce a different response and may trigger an immune response, for example, resulting in changes in the arterial waveform shape. The systems and methods described herein can be used to detect such changes using arterial Doppler waveforms in order to screen patients for medical disorders or conditions.

### CROSS-REFERENCE TO RELATED APPLICATIONS

This application claims the benefit of U.S. provisional patent application No. 63/059,242, filed on Jul. 31, 2020, and titled “SYSTEMS AND METHODS FOR ASSESSING INTERNAL LUMEN SHAPE CHANGES TO SCREEN PATIENTS FOR A MEDICAL DISORDER OR DISEASE,” the disclosure of which is expressly incorporated herein by reference in its entirety.

### BACKGROUND

Currently, there are a number of solutions for diagnosis of medical disorders or conditions. Many of these solutions include multiple tests done in hospital and medical testing centers, and such testing can be expensive and frequently yield negative results.

The internal luminal shape of a person's blood vessels is unique, and this shape changes as the person's physiology and anatomy changes with disease or with pathophysiological change. This change in shape affects the blood flow in the person's arteries. This change also affects the shape of the Doppler arterial waveform. Information about the shape is contained in the frequency components of the Doppler arterial waveform. Factors that cause shape change in the internal lumen of the blood vessels include blockages, aneurysms, vasodilator substances, and vasoconstrictor substances. There are many of these endogenous vasodilators (substances made in the body), for example, in response to diseases or disorder.

It would be advantageous to provide an immediate screening test for diseases or disorders that cause a change in the internal luminal shape of a person's blood vessels and therefore arterial waveform.

### SUMMARY

Systems and methods for assessing internal lumen shape changes to screen patients for a medical disorder or condition are described herein. An example method includes receiving an arterial Doppler signal for a target patient; converting the arterial Doppler signal into a frequency domain; and analyzing the frequency-domain arterial Doppler signal to identify one or more features. The method also includes comparing the one or more features of the frequency-domain arterial Doppler signal to a library, where the library includes respective arterial Doppler signal data and respective clinical data for a plurality of historical patients. The method further includes screening the target patient for a medical disorder or disease based on the comparison, where the medical disorder or disease causes vasodilation or vasoconstriction of the target patient's arteries.

Additionally, the one or more features includes a frequency component, an amplitude of the frequency component, a phase of the frequency component, and/or a power spectrum.

Alternatively or additionally, the one or more features include respective Fourier coefficients associated with a plurality of harmonics of the frequency-domain arterial Doppler signal.

Alternatively or additionally, the one or more features include a shape of the frequency-domain arterial Doppler signal.

In some implementations, the step of comparing the one or more features of the frequency-domain arterial Doppler signal to the library includes performing a statistical analysis. Optionally, the statistical analysis yields a probability score for a presence of the medical disorder or disease in the target patient.

In some implementations, the step of comparing the one or more features of the frequency-domain arterial Doppler signal to the library includes recognizing a pattern in the frequency-domain arterial Doppler signal and/or the one or more features; and correlating the frequency-domain arterial Doppler signal and/or the one or more features with one or more of the respective arterial Doppler signal data for the historical patients stored in the library based the recognized pattern. For example, the target patient can be screened for the medical disorder or disease based on the respective clinical data associated with the one or more of the respective arterial Doppler signal data for the historical patients stored in the library.

In some implementations, the step of comparing the one or more features of the frequency-domain arterial Doppler signal to the library includes inputting the frequency-domain arterial Doppler signal and/or the one or more features into a machine learning module, where the machine learning module is configured to screen the target patient for the medical disorder or disease.

In some implementations, the method further includes maintaining the library. The step of maintaining the library optionally includes receiving a plurality of respective arterial Doppler signals and respective clinical data for a plurality of historical patients; converting the respective arterial Doppler signals for the historical patients into the frequency domain; analyzing each of the respective frequency-domain arterial Doppler signals for the historical patients to identify one or more features; and associating the one or more features of the respective frequency-domain arterial Doppler signals for the historical patients with the respective clinical data for each of the historical patients.

Alternatively or additionally, the arterial Doppler signal is converted into the frequency domain using a Laplace transform, a Fourier transform, a discrete Fourier transform, or a z-transform.

Alternatively or additionally, in some implementations, the arterial Doppler signal is a digital signal. In other implementations, the arterial Doppler signal is an analog signal.

Alternatively or additionally, the arterial Doppler signal is obtained from the target patient's radial, carotid, femoral, or brachial artery.

In some implementations, the medical disorder or disease causes vasodilation or vasoconstriction of the target patient's arteries. In some implementations, the medical disorder or disease is a viral or bacterial infection. In some implementations, the medical disorder or disease is sepsis.

An example system for screening patients for a medical disorder or condition based on an arterial Doppler signal is also described herein. The system includes a handheld ultrasound probe; and a computing device operably coupled to the handheld ultrasound probe. The computing device includes a processor and a memory operably coupled to the processor, the memory having computer-executable instructions stored thereon. The computing device is configured to receive an arterial Doppler signal for a target patient; convert the arterial Doppler signal into a frequency domain; and analyze the frequency-domain arterial Doppler signal to identify one or more features. The computing device is also configured to compare the one or more features of the frequency-domain arterial Doppler signal to a library, where the library includes respective arterial Doppler signal data and respective clinical data for a plurality of historical patients. The computing device is further configured to screen the target patient for a medical disorder or disease based on the comparison, where the medical disorder or disease causes vasodilation or vasoconstriction of the target patient's arteries.

Additionally, the system optionally further includes a handheld computing device operably coupled to the handheld ultrasound probe. The handheld computing device is configured to receive the arterial Doppler signal for the target patient from the handheld ultrasound probe; and transmit the arterial Doppler signal for the target patient to the computing device. Optionally, the handheld computing device is a smartphone or a tablet.

Another method for screening patients for arterial disease includes receiving an arterial Doppler signal for a target patient; converting the arterial Doppler signal into a frequency domain; and analyzing the frequency-domain arterial Doppler signal to identify one or more features. The method also includes comparing the one or more features of the frequency-domain arterial Doppler signal to a library, where the library includes respective arterial Doppler signal data and respective clinical data for a plurality of historical patients. The method further includes screening the target patient for arterial disease based on the comparison. Optionally, the method further includes recommending diagnostic testing. Optionally, the method further includes recommending a medical procedure.

In some implementations, the arterial disease is atherosclerosis. In other implementations, the arterial disease is an aneurysm.

Alternatively or additionally, the method further includes recommending a medical procedure such as stent insertion.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a diagram illustrating a system for screening patients for a medical disorder or disease based on an arterial Doppler signal according to implementations described herein.

FIG. 2 is a flowchart illustrating example operations for screening patients for a medical disorder or disease based on an arterial Doppler signal according to implementations described herein.

FIG. 3 is an example computing device.

FIGS. 4A and 4B illustrate the internal lumen shape of healthy (FIG. 4A) and unhealthy (FIG. 4B) arteries.

FIG. 5 is graphical display of an example system configured to perform Doppler ultrasound.

FIG. 6 illustrates example blood flow dynamics (shear stress) in proximity to a fusiform aneurysm.

FIG. 7 illustrates example blood flow dynamics (velocity) in proximity to a saccular aneurysm.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for screening patients for a medical disorder or disease that causes vasodilation or vasoconstriction of arteries, it will become evident to those skilled in the art that the implementations are not limited thereto, but are applicable for screening patients for other medical disorders or conditions such as arterial disease.

The systems and methods described herein can be used for assessing internal lumen shape changes of the blood vessels to screen patients for a medical disorder or condition. Factors that cause shape change in the internal lumen of the blood vessels include, but are not limited to, partial blockage due to atherosclerosis or plaque deposit in the wall of arteries (which causes narrowing of the arterial lumen), aneurysms causing bulging of the arterial wall, presence of vasodilators substances, or presence of vasoconstrictor substances. Change in shape of the internal lumen of arteries results in a change in the Doppler waveform shape obtained from arteries. As described below, computerized mathematical analysis of the Doppler waveform is used to provide a probability estimate for the presence of a medical disorder or disease in a patient.

All infectious diseases have stages from mild infection to severe infection with many stages in between. For example, infections typically have four stages: incubation, prodromal, illness, and convalescence. The incubation stage begins the moment when the pathogen enters the body and ends with the appearance of the first symptoms. In the prodromal stage, non-specific symptoms appear first, like fever, tiredness and general discomfort. This stage usually lasts a few days. In the illness stage, more specific symptoms appear. The number of pathogens is at its peak here too. The manifestations and length will depend on the patient and the disease. Finally, in the convalescence stage, symptoms disappear, and the immune system returns to normal.

Each particular infectious organism and infectious state is expected to produce a different response and may trigger an immune response, for example, resulting in changes in the luminal shape of a person's blood vessels and therefore arterial waveform shape. This is due, at least in part, to release of vasodilator substances into the blood vessels. This disclosure contemplates that changes in the arterial waveform shape may be categorized for each stage. In other words, each stage of infection may have a unique response and some of these responses may produce a specific signal detectable using the methods described herein.

Waveforms such as arterial Doppler waveforms contain physiologic and pathophysiologic information. Arterial Doppler waveforms refer to the morphology of pulsatile blood flow velocity tracings on spectral Doppler ultrasound. Arterial Doppler waveforms capture the different phases of arterial flow: rapid antegrade flow reaching a peak during systole, transient reversal of flow during early diastole, and slow antegrade flow during late diastole. Arterial waveforms are derived from pressure transducers, ultrasound devices and other body scanners. Mathematical analysis (e.g., feature extraction, statistics, multivariate analysis such as principal component analysis (PCA), etc.) can access information contained in arterial waveforms and help to convert it into useful data and knowledge about disease states. In order to convert this information into knowledge, a library (or libraries) of arterial waveforms (or data representing arterial waveforms) from a plurality of patients, their mathematical analysis, and clinical information can be created. Arterial waveforms for new patients (or data representing arterial waveforms for new patients) can be compared against such library. Creating a library can be accomplished by collecting arterial waveforms from historical patients with known medical conditions (e.g., which is contained in the clinical data associated with historical patients) and performing the mathematical analysis of the signals to find the unique disease signatures or markers in the signals. Such markers can be used to identify the disease in future patients.

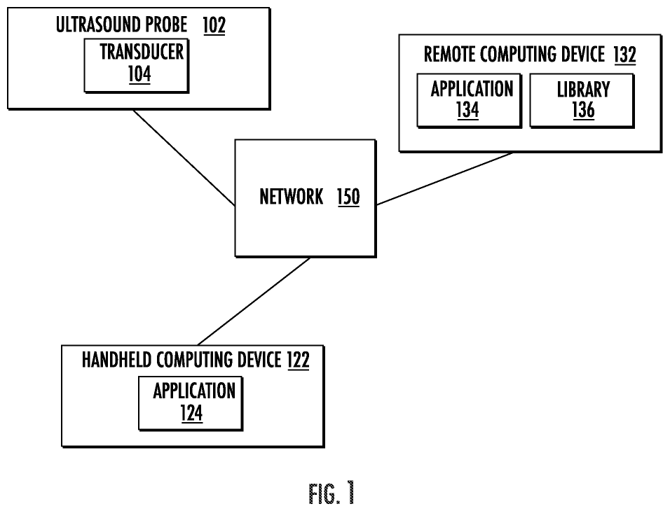

Referring now to FIG. 1, an example system for screening patients for a medical disorder or disease based on an arterial Doppler signal is shown. The term “patient” is defined herein to include animals such as mammals, including, but not limited to, primates (e.g., humans), cows, sheep, goats, horses, dogs, cats, rabbits, rats, mice and the like. In some implementations, the patient is a human. This disclosure contemplates that the methods for screening patients for a medical disorder or disease based on an arterial Doppler signal can be performed using the system shown in FIG. 1. The methods described herein are non-invasive and provide a means to create pathophysiologic data and knowledge. Additionally, the methods described herein provide a non-invasive means to find unique signals for medical conditions. The methods described herein also provide an immediate screening test for diseases or disorders that cause a change in the shape of the arterial Doppler waveform. These methods provide an improvement over existing diagnostic testing, which can be expensive and frequently yield negative results. The system includes an ultrasound probe 102, a handheld computing device 122, and a remote computing device 132. It should be understood that the methods for screening patients for a medical disorder or disease based on an arterial Doppler signal described herein may be performed using a computing environment having more or less components and/or with components arranged differently than shown in FIG. 1.

The ultrasound probe 102, the handheld computing device 122, and the remote computing device 132 are operably coupled to one or more networks 150. This disclosure contemplates that the networks 150 are any suitable communication network. The networks 150 can be similar to each other in one or more respects. Alternatively or additionally, the networks 150 can be different from each other in one or more respects. The networks 150 can include a local area network (LAN), a wireless local area network (WLAN), a wide area network (WAN), a metropolitan area network (MAN), a virtual private network (VPN), etc., including portions or combinations of any of the above networks. Additionally, each of the ultrasound probe 102, the handheld computing device 122, and the remote computing device 132 are coupled to the one or more networks 150 through one or more communication links. This disclosure contemplates the communication links are any suitable communication link. For example, a communication link may be implemented by any medium that facilitates data exchange including, but not limited to, wired, wireless and optical links. Example communication links include, but are not limited to, a LAN, a WAN, a MAN, Ethernet, the Internet, or any other wired or wireless link such as WiFi, WiMax, 3G, 4G, or 5G.

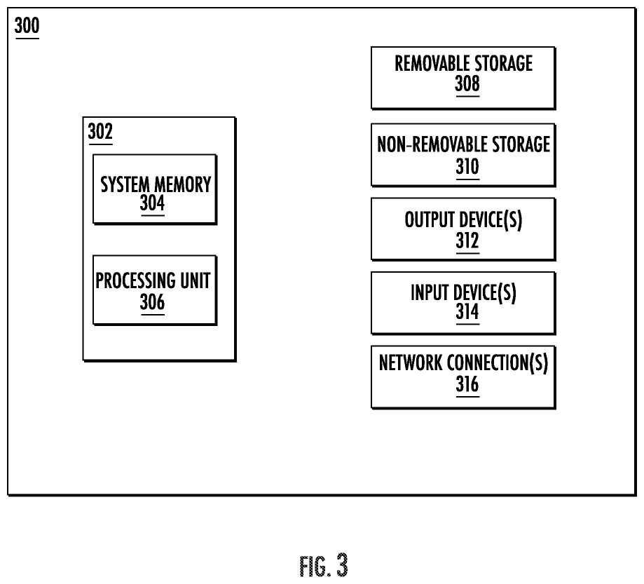

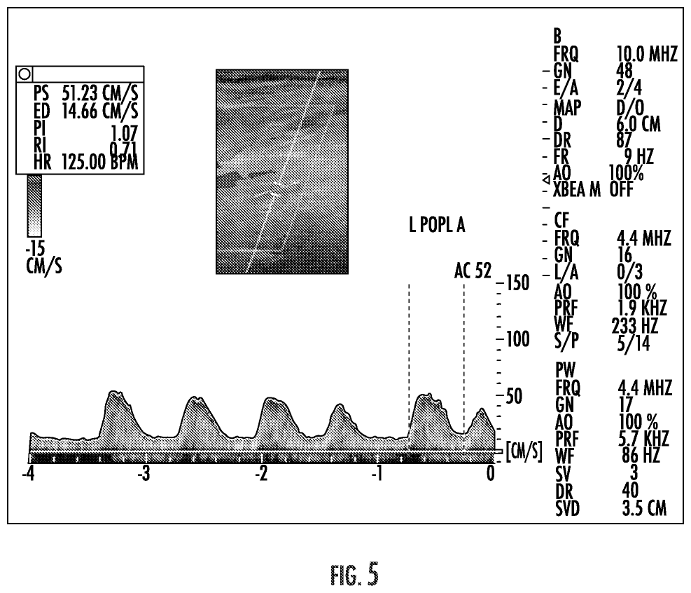

The ultrasound probe 102 includes a transducer 104 that generates sound waves, which reflect from anatomical features (e.g., body tissue) when delivered into the body, and receives the resulting echoes. The echoes are subsequently analyzed by a computing device. This disclosure contemplates that such computing device can be the computing device shown in FIG. 3. Additionally, such computing device can be incorporated into the ultrasound probe 102 or operably coupled to the ultrasound probe (e.g., the handheld computing device 122 and/or the remote computing device 132). Ultrasound probes may be used for ultrasound imaging applications, where a computing device analyzes the echoes to produce an image (i.e., sonogram). Alternatively or additionally, ultrasound probes can be used for Doppler ultrasound applications, where a computing device evaluates movement of material (e.g., blood flow) within a body. The ultrasound probe 102 described herein is configured for Doppler ultrasound applications. A graphical display of an example system configured to perform Doppler ultrasound is shown in FIG. 5. Additionally, the ultrasound probe 102 may be a vascular probe. Optionally, the ultrasound probe 102 may be a handheld or portable ultrasound probe. Ultrasound probes are known in the art and therefore not described in further detail herein. In some implementations, the ultrasound probe 102 is configured to transmit the arterial Doppler signals to the remote computing device 132 for further processing. In other implementations, the ultrasound probe 102 is configured to transmit the arterial Doppler signals to the handheld computing device 122 for further processing.

The handheld computing device 122 can be a computing device such as the computing device shown in FIG. 3. The handheld computing device 122 may be a portable computing device associated with a user such as a laptop, tablet, smartphone, etc. The handheld computing device 122 can be configured to execute an application 124. The application 124 may be an application for interacting with data collected by the ultrasound probe 102. Optionally, as described above, the application 124 may be configured to analyze the echoes (e.g., Doppler ultrasound application). Alternatively or additionally, in some implementations, the handheld computing device 122 can be configured to receive (and optionally store) arterial Doppler signals from the ultrasound probe 102, and transmit the arterial Doppler signals to the remote computing device 132 for further processing. Alternatively or additionally, in some implementations, the handheld computing device 122 can be configured to process the arterial Doppler signals, which can include, but is not limited to, analog-to-digital conversion, frequency domain transformation, feature identification, and/or data analysis (e.g., statistical analysis).

The remote computing device 132 can be a computing device such as the computing device shown in FIG. 3. The remote computing device 132 may optionally be a server computing device. Alternatively or additionally, the remote computing device 132 may optionally be a computing cluster, e.g., a plurality of computing devices. A computing cluster is a distributed computing environment where tasks are performed by computing devices that are linked through a communication network or other data transmission medium. The remote computing device 132 can be configured to execute an application 134. The application 134 may be an application for interacting with data collected by the ultrasound probe 102 and/or the handheld computing device 122. Optionally, as described above, the application 134 may be configured to analyze the echoes (e.g., Doppler ultrasound application), perform analog-to-digital conversion, perform frequency domain transformation, perform feature identification, and/or perform data analysis (e.g., statistical analysis). Alternatively or additionally, the remote computing device 132 can optionally maintain a library 136. As described herein, the library 136 may include a plurality of respective arterial Doppler signals and respective clinical data (which includes diagnosis of a medical disorder or disease) for a plurality of historical patients.

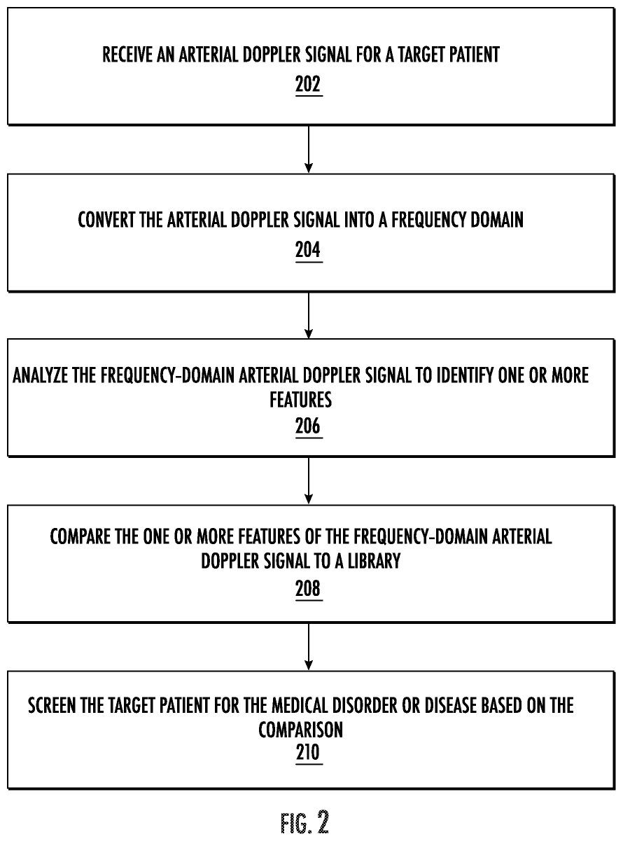

Referring now to FIG. 2, a flowchart illustrating example operations for screening patients for a medical disorder or disease is shown. In FIG. 2, the medical disorder or disease causes vasodilation or vasoconstriction of the target patient's arteries. In some implementations, the medical disorder or disease is a viral or bacterial infection. For example, the medical disorder or disease may be a virus such as the novel coronavirus 19 (COVID-19) also known as the severe acute respiratory syndrome coronavirus 2 (SARS-CoV-2). This disclosure contemplates that a virus such as COVID-19 may cause release of chemicals into tissues and/or into the blood stream that may change the shape of the internal lumen shape, resulting in a change in the arterial waveform, and so produce a change in the target patient's normal arterial Doppler signal. Such change may be detectable in the prodromal and/or illness stages as discussed above and the change may be indicative of the virus. Alternatively or additionally, the change in shape of the arterial waveform may be used to inform treatment (e.g., recommend further diagnostic testing). Alternatively, the medical disorder or disease may be sepsis. Sepsis causes vascular collapse meaning severe vasodilation and low blood pressure and so produce a change in the target patient's normal arterial Doppler signal. Such change may be indicative of the infection. Mediators of sepsis are released by the immune system and cause vascular system changes. Such change may be detectable in the prodromal and/or illness stages as discussed above and the change may be indicative of the infection. Alternatively or additionally, the change in shape of the arterial waveform may be used to inform treatment (e.g., recommend further diagnostic testing). This disclosure contemplates that the operations shown in FIG. 2 can be performed by a computing device such as the handheld computing device 122 and/or the remote computing device 132 shown in FIG. 1. In some implementations, the operations shown in FIG. 2 may be performed entirely by the handheld computing device 122 shown in FIG. 1. In other implementations, the operations shown in FIG. 2 may be performed entirely by the remote computing device 132 shown in FIG. 1. In yet other implementations, the operations shown in FIG. 2 may be performed by a combination of the handheld computing device 122 and the remote computing device 132 shown in FIG. 1.

At step 202, an arterial Doppler signal for a target patient is received. The arterial Doppler signal can be collected, for example, using the ultrasound probe 102 shown in FIG. 1. For example, the ultrasound probe is sent into the blood vessel, and the reflected pulse is received and analyzed to produce a waveform (also referred to herein as “arterial Doppler signal” or “arterial Doppler waveform”) which represents the velocity of the blood flowing in the blood vessel. The shape of arterial Doppler waveforms refer to blood flow velocity tracings. Such waveforms differ by the vascular bed (peripheral, cerebrovascular, and visceral circulations) and/or the presence of medial disorder or disease. As described above, the ultrasound probe can transmit Doppler signals to the handheld computing device 122 and/or the remote computing device 132 shown in FIG. 1. The arterial Doppler signal can be obtained from the target patient's artery. Arteries include, but are not limited to, the radial, carotid, femoral, or brachial artery. In some implementations, the arterial Doppler signal is a digital signal. In other implementations, the arterial Doppler signal is an analog signal. Optionally, an analog signal can be converted to a digital signal. This disclosure contemplates that the ultrasound probe may be configured to perform analog-to-digital conversion (ADC) in some implementations, while in other implementations this processing is performed by another computing device. ADC techniques are well known in the art and therefore not described in further detail herein.

Referring again to FIG. 2, at step 204, the arterial Doppler signal, which is in the time domain, is converted into a frequency domain. Techniques for converting time domain signals into the frequency domain are known in the art and include, but are not limited to, a Laplace transform, a Fourier transform, a discrete Fourier transform, a fast Fourier transform, or a z-transform. This disclosure contemplates using any known technique for converting time domain signals into the frequency domain. For example, the Fourier analysis is used to convert a signal from its original domain (e.g., time domain) into a frequency domain representation and vice versa. Fourier analysis is based on the premise that certain signals (e.g., continuous or discrete and periodic or aperiodic signals) can be represented by a sum of sinusoids. In other words, certain signals can be decomposed into various sine and cosine waveforms. One skilled in the art would appreciate that analyzing sinusoidal components is easier and/or more efficient than analyzing the original signal. Eqn. (1) below illustrates the Fourier series of an arterial Doppler signal:

$$f ( t ) = {a}_{0} + {\sum }_{n = 1}^{∞} ( {a}_{n} cos \frac{n π t}{T} + {b}_{n} sin \frac{n π t}{T} ) , \qquad ( 1 )$$

where a₀ is a constant, aₙ and bₙ are the Fourier coefficients, n is the number of harmonics, and T is the fundamental period. One skilled in the art would appreciate that aₙ and bₙ are represent relative weights (which effect amplitude) of the nth harmonic.

At step 206, the frequency-domain arterial Doppler signal is analyzed to identify one or more features. In some implementations, the one or more features include a frequency component and its amplitude. As described above, converting the arterial Doppler signal into the frequency domain decomposes the arterial Doppler signal into a plurality of frequency components, each being associated with a respective amplitude. Optionally, this information can be stored as a histogram or bar graph representing the intensity of each frequency component in the signal. In some implementations, one or more features are identified for each of 10 harmonics. It should be understood that 10 harmonics is provided only as an example. This disclosure contemplates identifying one or more features for each of 2, 3, 4, 5, 6, 7, 8, 9, 10, 11, 12, 13, 14, 15, 16, 17, 18, 19, 20 or more harmonics in other implementations. For Fourier analysis, the frequency components are sine and/or cosine waveforms for a plurality of harmonics, where each sinusoid has an amplitude. The arterial Doppler signal is a sum of the sinusoids for thee plurality of harmonics. As described below, the frequency components and respective amplitudes (e.g., spectrum) for the plurality of harmonics associated with the arterial Doppler signal can serve as a signature or marker for the medical disorder or disease. It should be understood that the frequency components and respective amplitudes are provided only as example features. This disclosure contemplates that the one or more features may include, but are not limited to, a phase of the frequency component, a shape of the frequency-domain arterial Doppler signal, and/or a power spectrum. The phase of the frequency component, shape of the frequency-domain arterial Doppler signal, and/or power spectrum associated with the arterial Doppler signal can serve as a signature or marker for the medical disorder or disease.

In some implementations, the one or more features include respective Fourier coefficients associated with a plurality of harmonics of the frequency-domain arterial Doppler signal. As described above, for Fourier analysis, the frequency components are sine and/or cosine waveforms for a plurality of harmonics (see Eqn. (1) above). The Fourier coefficients represent relative weights associated with the sinusoids of the harmonics. In other words, when the arterial Doppler signal is converted into a frequency domain using a Fourier transform (discrete Fourier transform or fast Fourier transform for digital signals), the transformation yields Fourier coefficients. The Fourier coefficients for the plurality of harmonics associated with the arterial Doppler signal can serve as a signature or marker for the medical disorder or disease. For example, in some implementations, Fourier coefficients are identified for each of 10 harmonics. It should be understood that 10 harmonics is provided only as an example. This disclosure contemplates identifying Fourier coefficients for each of 2, 3, 4, 5, 6, 7, 8, 9, 10, 11, 12, 13, 14, 15, 16, 17, 18, 19, 20 or more harmonics in other implementations.

At 208, the one or more features of the frequency-domain arterial Doppler signal are compared to a library. As described below, the step of comparing may include a statistical analysis or modelling. The library may include respective arterial Doppler signal data and respective clinical data for a plurality of historical patients. As described herein, the library may be maintained by the remote computing device 132 shown in FIG. 1 (e.g., library 136). The step of maintaining the library optionally includes receiving a plurality of respective arterial Doppler signals and respective clinical data for a plurality of historical patients; converting the respective arterial Doppler signals for the historical patients into the frequency domain; and analyzing each of the respective frequency-domain arterial Doppler signals for the historical patients to identify one or more features. It should be understood that the respective arterial Doppler signals for the historical patients can be processed in the same manner as described with respect to steps 202-206 of FIG. 2. As a result, the respective features (such as frequency components and amplitudes, Fourier coefficients, etc.) associated with an arterial Doppler signals for each of the historical patients are identified. The step of maintaining the library optionally further includes associating the one or more features (such as frequency components and amplitudes, Fourier coefficients, etc.) of the respective frequency-domain arterial Doppler signals for the historical patients with the respective clinical data for each of the historical patients. It should be understood that the respective clinical data includes whether a historical patient has been diagnosed with a medical disorder or disease (e.g., a viral or bacterial infection, sepsis, arterial disease, or other disorder or disease that causes vasodilation or vasoconstriction of the arteries), as well as the state of the medical disorder or disease. In this way, the features (such as frequency components and amplitudes, Fourier coefficients, etc.) can serve as a signature or marker for the medical disorder or disease and/or the state of such medical disorder or disease.

In some implementations, the step of comparing the one or more features of the frequency-domain arterial Doppler signal to the library includes performing a statistical analysis. Optionally, in some implementations, the statistical analysis is a multivariate analysis such as principal component analysis (PCA). This disclosure contemplates using any statistical analysis known in the art. The statistical analysis involves analyzing the one or more features of the frequency-domain arterial Doppler signal for the target patient in relation to the features for the historical patients, which are stored in the library. Such a statistical analysis yields a probability score for a presence of the medical disorder or disease in the target patient. In other words, the statistical analysis determines how closely the one or more features of the frequency-domain arterial Doppler signal for the target patient are related those of historical patients having the medical disorder or disease.

In one aspect, the one or more features of the frequency-domain arterial Doppler signal for the target patient is the spectrum of frequencies and corresponding amplitudes the frequency-domain arterial Doppler signal for the target patient (e.g., frequency components and amplitudes). This information can be compared to the respective spectra of frequencies and amplitudes of the arterial Doppler waveforms for the historical patients, which are associated with specific medical disorders or diseases, stored in the library. The comparison can yield a probability score for a presence of the medical disorder or disease in the target patient. This result gives the screened patient and medical team the confidence that more expensive and definitive diagnostic testing is worthwhile.

In another aspect, the one or more features of the frequency-domain arterial Doppler signal for the target patient are Fourier coefficients associated with a plurality of harmonics of the frequency-domain arterial Doppler signal for the target patient. This information can be compared to the respective Fourier coefficients associated with a plurality of harmonics of the arterial Doppler waveforms for the historical patients, which are associated with specific medical disorders or diseases, stored in the library. The comparison can yield a probability score for a presence of the medical disorder or disease in the target patient. This result gives the screened patient and medical team the confidence that more expensive and definitive diagnostic testing is worthwhile.

In some implementations, the step of comparing the one or more features of the frequency-domain arterial Doppler signal to the library includes recognizing a pattern in the frequency-domain arterial Doppler signal and/or the one or more features; and correlating the frequency-domain arterial Doppler signal and/or the one or more features with one or more of the respective arterial Doppler signal data for the historical patients stored in the library based the recognized pattern.

In some implementations, the step of comparing the one or more features of the frequency-domain arterial Doppler signal to the library includes inputting the frequency-domain arterial Doppler signal and/or the one or more features into a machine learning module, where the machine learning module is configured to screen the target patient for the medical disorder or disease. Machine learning models map inputs (e.g., model features such as the one or features for the target patient) to outputs (e.g., model targets such as a prediction of medical disorder or disease). Machine learning models ‘learn’ such mapping through training. It should be understood that machine learning models may be supervised (i.e., require labeled data), unsupervised (i.e., do not require labeled data), or semi-supervised. This disclosure contemplates that the machine learning module may be any supervised, unsupervised, or semi-supervised learning model. For example, in some implementations, the machine learning model may be an artificial neural network, which is trained on the data stored in the library to predict presence of the medical disorder or disease. In these implementations, the one or more features (e.g., frequency components and amplitudes, Fourier coefficients associated with a plurality of harmonics, or other features) for the target patient (i.e., model features) are input into a trained artificial neural network, which predicts presence of the medical disorder or disease in the target patient (i.e., model target). Machine learning models and training are known in the art and therefore not described in further detail herein.

Referring again to FIG. 2, at step 210, the target patient is screened for the medical disorder or disease based on the comparison. As used herein, screening is identifying or detecting that a patient may have an unrecognized medical disorder or disease. Screening is different than diagnosing a patient with the medical disorder or disease. It should be understood that screening has higher risk of false positive/negative than diagnosis. For example, the objective of screening is to identify a patient that may benefit for further diagnostic testing. Optionally, the step of screening can include providing a probability that the target patient has the medical disorder or disease. This includes providing a probability that the target patient has the medical disorder or disease of a certain stage (e.g., incubation, prodromal, illness, and convalescence stages). This disclosure contemplates that the one or more features of the arterial Doppler signal can serve as a signature or marker associate with both medical disorders or diseases as well as stages thereof. In other words, this disclosure contemplates that the one or more features of the arterial Doppler signal change with disorder or disease and/or stage thereof.

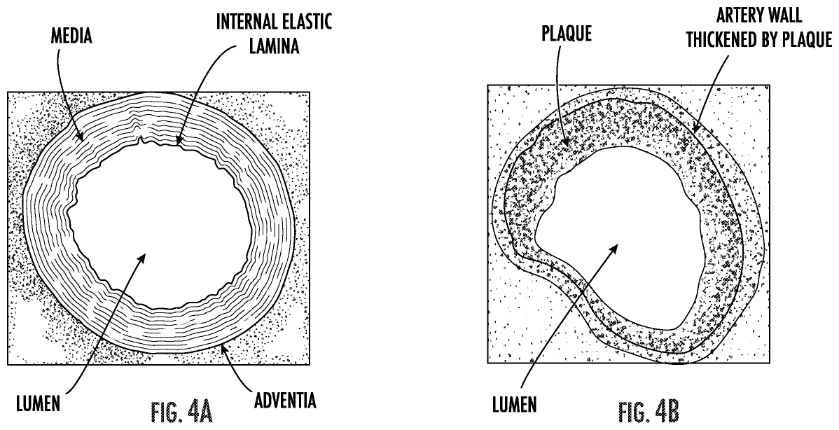

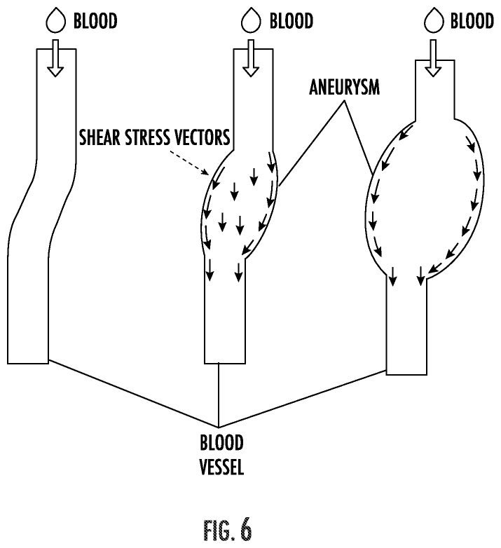

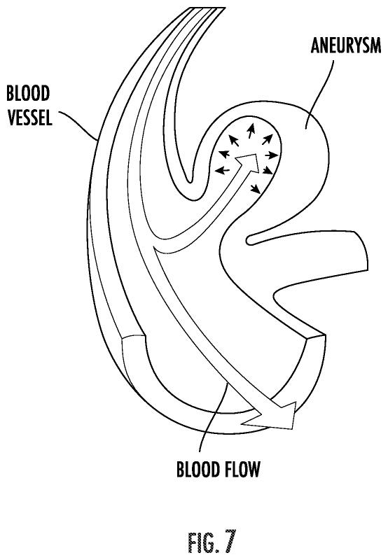

As described above, the example operations for screening patients for a medical disorder or disease shown in FIG. 2 are directed to a medical disorder or disease that causes vasodilation or vasoconstriction of the target patient's arteries. This disclosure contemplates that patients may be screened for other medical disorders or diseases based on based on an arterial Doppler signal. Other medical disorders or diseases may include, but are not limited to, arterial disease. As used herein, arterial diseases is any abnormal arterial condition including, but not limited to, obstructions (e.g., atheromatous plaques—see FIGS. 4A and 4B) and aneurysm (e.g., abdominal, femoral, cerebral—see FIGS. 6 and 7).

Arterial Disease (such as atherosclerosis, aneurysms) changes the shape of the internal lumen of arteries. Those skilled in the art would appreciate that diagnostic testing of arteries such as arteriograms, computed tomography (CT), and magnetic resonance imaging (MRI) exams look at the artery from outside the body to the inside and generate an image of the vessels and their pathologies. In contrast, arterial Doppler waveforms capture information about blood flowing through the patient's vessels. When blood flows in arteries, the velocity of flow may change when pathology in the vessel is encountered by the blood. For example, when blood flows past a partial atherosclerotic obstruction of the femoral artery, the velocity of the blood entering the obstructed area changes the velocity of the blood entering the obstruction and velocity of blood passing the obstruction and velocity of blood leaving the obstruction. The blood flow can be thought of as an information carrier, and this information is characteristic for the particular pathology. A single red blood cell will have its velocity changed as it encounters/passes the pathology, and the pathological process imparts new information to the velocity of red cells as they encounters/passes the pathology. This information may be indirectly accessed by arterial Doppler waveforms. Therefore, analyzing arterial Doppler waveforms in the frequency domain as described herein yields a set of harmonics that are characteristic for the particular pathology.

An aneurysm is a bulge in an artery that develops in areas where the vessel wall is weak. Aneurysms can occur in all arteries (including Aorta, Cerebral arteries, femoral arteries). Rupture of aneurysms have potential devastating consequences for the patient, patient's family and society. Blood flow patterns are changed when blood flows into and past an aneurysm, and this change in blood flow can be thought of as changing information about the vessel. Such a change in blood flow pattern has frequency components that are characteristic for the aneurysm.

As blood flows through arteries it carries information about the state of the artery, the velocity of blood changes as blood encounters arterial pathologies (e.g., including but not limited to atherosclerotic obstructions, aneurysmal dilation of vessels), which produce changes blood flow patterns. One method to access this information is using harmonic analysis of the arterial Doppler waveforms. As described herein, a library storing sets of harmonics for each individual disease and/or state of disease can be collected and using such a library the arterial Doppler waveform from an individual can be compared with the library to generate a probability estimate of disease or disease state. This probability estimate can be used as a screening tool for arterial disease.

Distinguishing differences in shapes of the internal lumen of arteries using harmonic analysis of arterial Doppler waveforms can produce signatures of disease conditions. Having this signature allows screening for conditions such as cerebral aneurysm or other vascular condition that changes the internal shape of the lumen of arteries. Using the systems and methods described herein to identify signatures for diseases and/or disease states that cause shape changes of arteries and using those signatures to screen for arterial disease provides an opportunity for early treatment that may significantly reduce the consequences of these disease states.

Common arterial diseases are stroke, peripheral artery disease (PAD), abdominal aortic aneurysm (AAA), carotid artery disease (CAD), arteriovenous malformation (AVM), critical limb ischemia (CLI), pulmonary embolism (blood clots), deep vein thrombosis (DVT), chronic venous insufficiency (CVI), and varicose veins. Optionally, the patients may be diagnosed with the other medical disorders or diseases. A method for screening patients for arterial disease may include receiving an arterial Doppler signal for a target patient; converting the arterial Doppler signal into a frequency domain; and analyzing the frequency-domain arterial Doppler signal to identify one or more features. The method also includes comparing the one or more features of the frequency-domain arterial Doppler signal to a library, where the library includes respective arterial Doppler signal data and respective clinical data for a plurality of historical patients. The method further includes screening the target patient for arterial disease (e.g., atherosclerosis or aneurysm) based on the comparison. Optionally, the method further includes recommending (and optionally performing) further definitive diagnostic testing. Optionally, the method further includes recommending (and optionally performing) a medical procedure. For example, the method can be used to screen for aneurysms. Those skilled in the art would appreciate that diagnosis of aneurysms may require medical imaging (e.g., MRI), which is expensive. For example, a patient may report slight headache during an emergency visit. While most strokes are caused by clots, some strokes are caused by bleeds. The headache may be a symptom of an aneurysm in danger of rupture; however, the patient may forego recommended diagnostic imaging due to cost (and this decision may be devastating for the patient). Those skilled in the art would appreciate that time is of essence when treating aneurysms. The systems and methods described herein can therefore screen the target patient and as a result recommend medical treatment such as stent insertion (e.g., flow diversion) to treat an aneurysm before it ruptures.

It should be appreciated that the logical operations described herein with respect to the various figures may be implemented (1) as a sequence of computer implemented acts or program modules (i.e., software) running on a computing device (e.g., the computing device described in FIG. 3), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 3, an example computing device 300 upon which the methods described herein may be implemented is illustrated. It should be understood that the example computing device 300 is only one example of a suitable computing environment upon which the methods described herein may be implemented. Optionally, the computing device 300 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 300 typically includes at least one processing unit 306 and system memory 304. Depending on the exact configuration and type of computing device, system memory 304 may be volatile (such as random access memory (RAM)), non-volatile (such as read-only memory (ROM), flash memory, etc.), or some combination of the two. This most basic configuration is illustrated in FIG. 3 by dashed line 302. The processing unit 306 may be a standard programmable processor that performs arithmetic and logic operations necessary for operation of the computing device 300. The computing device 300 may also include a bus or other communication mechanism for communicating information among various components of the computing device 300.

Computing device 300 may have additional features/functionality. For example, computing device 300 may include additional storage such as removable storage 308 and non-removable storage 310 including, but not limited to, magnetic or optical disks or tapes. Computing device 300 may also contain network connection(s) 316 that allow the device to communicate with other devices. Computing device 300 may also have input device(s) 314 such as a keyboard, mouse, touch screen, etc. Output device(s) 312 such as a display, speakers, printer, etc. may also be included. The additional devices may be connected to the bus in order to facilitate communication of data among the components of the computing device 300. All these devices are well known in the art and need not be discussed at length here.

The processing unit 306 may be configured to execute program code encoded in tangible, computer-readable media. Tangible, computer-readable media refers to any media that is capable of providing data that causes the computing device 300 (i.e., a machine) to operate in a particular fashion. Various computer-readable media may be utilized to provide instructions to the processing unit 306 for execution. Example tangible, computer-readable media may include, but is not limited to, volatile media, non-volatile media, removable media and non-removable media implemented in any method or technology for storage of information such as computer readable instructions, data structures, program modules or other data. System memory 304, removable storage 308, non-removable storage 310 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 306 may execute program code stored in the system memory 304. For example, the bus may carry data to the system memory 304, from which the processing unit 306 receives and executes instructions. The data received by the system memory 304 may optionally be stored on the removable storage 308 or the non-removable storage 310 before or after execution by the processing unit 306.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A computer-implemented method, comprising:
receiving an arterial Doppler signal for a target patient;
converting the arterial Doppler signal into a frequency domain;
analyzing the frequency-domain arterial Doppler signal to identify one or more features;
comparing the one or more features of the frequency-domain arterial Doppler signal to a library, the library comprising respective arterial Doppler signal data and respective clinical data for a plurality of historical patients; and
screening the target patient for a medical disorder or disease based on the comparison, wherein the medical disorder or disease causes vasodilation or vasoconstriction of the target patient's arteries.

2. The computer-implemented method of claim 1, wherein the one or more features comprise a frequency component, an amplitude of the frequency component, a phase of the frequency component, and/or a power spectrum.

3. The computer-implemented method of claim 1, wherein the one or more features comprise respective Fourier coefficients associated with a plurality of harmonics of the frequency-domain arterial Doppler signal.

4. The computer-implemented method of claim 1, wherein the one or more features comprise a shape of the frequency-domain arterial Doppler signal.

5. The computer-implemented method of claim 1, wherein comparing the one or more features of the frequency-domain arterial Doppler signal to the library comprises performing a statistical analysis.

6. The computer-implemented method of claim 5, wherein the statistical analysis yields a probability score for a presence of the medical disorder or disease in the target patient.

7. The computer-implemented method of claim 1, wherein the step of comparing the one or more features of the frequency-domain arterial Doppler signal to the library comprises:
recognizing a pattern in the frequency-domain arterial Doppler signal and/or the one or more features; and
correlating the frequency-domain arterial Doppler signal and/or the one or more features with one or more of the respective arterial Doppler signal data stored in the library based the recognized pattern.

8. The computer-implemented method of claim 7, wherein the target patient is screened for the medical disorder or disease based on the respective clinical data associated with the one or more of the respective arterial Doppler signal data stored in the library.

9. The computer-implemented method of claim 1, wherein the step of comparing the one or more features of the frequency-domain arterial Doppler signal to the library comprises inputting the one or more features of the frequency-domain arterial Doppler signal into a machine learning module, the machine learning module being configured to screen the target patient for the medical disorder or disease.

10. The computer-implemented method of claim 1, further comprising maintaining the library.

11. The computer-implemented method of claim 10, wherein the step of maintaining the library comprises:
receiving a plurality of respective arterial Doppler signals and respective clinical data for a plurality of historical patients;
converting the respective arterial Doppler signals for the historical patients into the frequency domain;
analyzing each of the respective frequency-domain arterial Doppler signals for the historical patients to identify one or more features; and
associating the one or more features of the respective frequency-domain arterial Doppler signals for the historical patients with the respective clinical data for each of the historical patients.

12. The computer-implemented method of claim 1, wherein the arterial Doppler signal is converted into the frequency domain using a Laplace transform, a Fourier transform, a discrete Fourier transform, a fast Fourier transform, or a z-transform.

13. The computer-implemented method of claim 1, wherein the arterial Doppler signal is a digital signal.

14. The computer-implemented method of claim 1, wherein the arterial Doppler signal is an analog signal.

15. The computer-implemented method of claim 1, wherein the arterial Doppler signal is obtained from the target patient's radial, carotid, femoral, or brachial artery.

16. The computer-implemented method of claim 1, wherein the medical disorder or disease is a viral or bacterial infection.

17. The computer-implemented method of claim 1, wherein the medical disorder or disease is sepsis.

18. A system, comprising:
a handheld ultrasound probe; and
a computing device operably coupled to the handheld ultrasound probe, the computing device comprising a processor and a memory operably coupled to the processor, the memory having computer-executable instructions stored thereon that, when executed by the processor, cause the processor to:
receive an arterial Doppler signal for a target patient;
convert the arterial Doppler signal into a frequency domain;
analyze the frequency-domain arterial Doppler signal to identify one or more features;
compare the one or more features of the frequency-domain arterial Doppler signal to a library, the library comprising respective arterial Doppler signal data and respective clinical data for a plurality of historical patients; and
screen the target patient for a medical disorder or disease based on the comparison, wherein the medical disorder or disease causes vasodilation or vasoconstriction of the target patient's arteries.

19. The system of claim 18, further comprising a handheld computing device operably coupled to the handheld ultrasound probe, wherein the handheld computing device is configured to:
receive the arterial Doppler signal for the target patient from the handheld ultrasound probe; and
transmit the arterial Doppler signal for the target patient to the computing device.

20. The system of claim 19, wherein the handheld computing device is a smartphone, a laptop, or a tablet.

21. A computer-implemented method, comprising:
receiving an arterial Doppler signal for a target patient;
converting the arterial Doppler signal into a frequency domain;
analyzing the frequency-domain arterial Doppler signal to identify one or more features;
comparing the one or more features of the frequency-domain arterial Doppler signal to a library, the library comprising respective arterial Doppler signal data and respective clinical data for a plurality of historical patients; and
screening the target patient for an arterial disease based on the comparison.

22. The computer-implemented method of claim 21, wherein the arterial disease is atherosclerosis.

23. The computer-implemented method of claim 21, wherein the arterial disease is an aneurysm.

24. The computer-implemented method of claim 21, further comprising recommending a medical procedure.

25. The computer-implemented method of claim 24, wherein the medical procedure is stent insertion.
